(19) United States
(12) Patent Application Publication
(10) Pub. No.: US 2004/0204433 A1
(43) Pub. Date: Oct. 14, 2004

(54) Hair growth stimulant compositions

(75) Inventors: Koji Imamura, Tokyo (JP); Rumi Ochiai, Tokyo (JP); Takako Okajima, Tokyo (JP); Susumu Morioka, Tokyo (JP); Taro Horie, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: TAISHO PHARMACEUTICAL CO., LTD.

(21) Appl. No.: 10/830,039

(22) Filed: Apr. 23, 2004

Related U.S. Application Data
(62) Division of application No. 10/240,654, filed on Oct. 3, 2002, now abandoned.
Application No. 10/240,654 is a 371 of international application No. PCT/JP01/02942, filed on Apr. 5, 2001.

(30) Foreign Application Priority Data
Apr. 7, 2000 (JP) 2000-106500

(51) Int. Cl. A61K 31/513

(52) U.S. Cl. 514/269

(57) ABSTRACT

Preparations containing minoxidil at a high concentration which scarcely show the precipitation of crystals even at low temperatures and have a high stability.

In preparations containing 10 to 50% by mass of water and 3% by mass or more of minoxidil, hair growth stimulant compositions further containing 8 to 30% of one or more polyhydric alcohols and having a pH value of 5.5 to 6.5 scarcely show precipitation of crystals and have a high stability.

### TECHNICAL FIELD

The present invention relates to a hair growth stimulant composition comprising minoxidil as an active component, and more particularly to a hair growth stimulant composition free from problems such as crystal precipitation and the like at low temperatures.

### BACKGROUND ART

The chemical name for minoxidil is 6-(1-piperidinyl)-2,4-pyrimidinediamine-3-oxide. The adaptation of minoxidil as a hair growth agent is disclosed in U.S. Pat. No. 4,139,619. Due to the excellent hair growth obtained by the topical application of minoxidil, hair growth agents containing minoxidil (hereinafter referred to as “minoxidil preparations”) have been widely accepted and their sales volume is record breaking.

To increase the effect of these minoxidil preparations, preparations having a higher concentration of minoxidil have been sought after. Until now, minoxidil preparations having a minoxidil concentration of about 3 to 5% by mass have been easily prepared (Japanese Patent Application Laid-open No. 150211/1988).

### DISCLOSURE OF THE INVENTION

However, when a high concentration minoxidil preparation (hereinafter referred to as “high conc. minoxidil preparation) contains 10 to 50% by mass of water in order to improve feeling during use, crystal precipitation occurs in the preparation if stored for a long period of time at a low temperature. Therefore, because redissolving the precipitated crystals takes time, problems occur when the hair growth composition is used in cold regions or when stored in cold places.

For this reason, a stable high conc. minoxidil preparation wherein crystal precipitation does not occur even if the preparation is stored at a low temperature has been required. Accordingly, an object of the present invention is to provide such a preparation.

As a result of diligent research on a solvent composition and the like of a high conc. minoxidil preparation to solve the above problems, the present inventors have discovered that by selecting a specific polyhydric alcohol and using it at a specific concentration as a solvent and adjusting the pH of the solution within a fixed range, aminoxidil solution in which a high concentration minoxidil is combined with a fixed amount of water does not precipitate crystals and minoxidil itself is stable, thereby completing the present invention.

Namely, an object of the present invention is to provide a hair growth stimulant composition comprising 10 to 50% by mass of water and 3% by mass or more of minoxidil, further comprising 8 to 30% by mass of one or more polyhydric alcohols, and having a pH of 5.5 to 6.5.

### DETAILED DESCRIPTION OF THE PREFERRED EMBODIMENT

The hair growth stimulant composition of the present invention contains 3% by mass or more (hereinafter simply referred to as “%”) of the active component minoxidil, with 3 to 6% being preferable.

As the solvent for dissolving the minoxidil, a mixture of polyhydric alcohol and water is used. It is required that the final composition comprises 10 to 50% of water and 8 to 30% of polyhydric alcohol. If either one of the polyhydric alcohol or water is not present, preventing the precipitation of crystals is difficult. Also, even if both are present, it is difficult to produce a composition that provides a good feeling during use and effective in suppressing the precipitation of crystals at low temperatures if they are present in an amount outside the above ranges. The present invention has a greater effect when the amount of water is 18% or more because the precipitation of crystals occurs easily. Polyhydric alcohol is incorporated in an amount according to the above range, with 10 to 20% being preferable, and 15 to 20% being particularly preferable.

It is desirable to add ethanol to the above solvent. The final composition preferably contains ethanol in an amount of 70% or less.

As the polyhydric alcohol used in the above solvent, 1,3-butylene glycol, glycerol, dipropylene glycol, Macrogol 400, Macrogol 600, and the like can be given, with 1,3-butylene glycol being preferable. Also, these polyhydric alcohols are preferably used in combinations of two or more types. As particularly preferable combinations of polyhydric alcohols, 1,3-butylene glycol and glycerol combined at a weight ratio of 10:1 to 1:4 and dipropylene glycol and glycerol combined at a weight ratio of 10:1 to 1:4 can be given.

The hair growth stimulant composition of the present invention is prepared by adding 3% or more of the active component minoxidil and other necessary active components and supplementary components to the solvent containing polyhydric alcohol and water, then mixing these components with stirring using a common method. In this instance, it is necessary to adjust the pH in a range of 5.5 to 6.5. If minoxidil is simply added to an aqueous type solvent, the resulting solution is neutral or weakly alkaline. When preparing the high conc. minoxidil preparation using this solution, precipitation of crystals occurs easily at a low temperature. On the other hand, if the solution is on the acid side with a pH of 5 or less, minoxidil itself becomes easily decomposable, even though precipitation of crystals does not occur.

The pH of the hair growth stimulant composition of the present invention is adjusted using a common method. As the pH adjustor, citric acid, hydrochloric acid, lactic acid, phosphoric acid, and the like are preferably used.

Furthermore, as medicinal components that are preferably added to and combined with the hair growth stimulant composition of the present invention, components selected from the group consisting of menthol, vitamin E acetate, pantothenylethylether, hinokitiol, glycyrrhetic acid, and diphenhydramine hydrochloride can be given (hereinafter referred to as “optional components”). Of these optional components, menthol possesses particularly high effectiveness. The addition of menthol as an essential component is even more preferable.

There are no particular limitations to the amount of these optional components to be added. This can be decided experimentally while taking the feeling during use and stability of minoxidil or solvent-type components into consideration. For example, the final composition preferably contains about 0.2 to 0.4% of menthol, a particularly preferable optional component.

In addition to the above components, the hair growth stimulant composition of the present invention may contain various active components and supplementary components normally used in topical preparations in an amount that does not harm the effect of the present invention. Examples of these components include fillers, vasodilators (carpronium chloride, benzyl nicotinate, swertia herb extract, panax ginseng extract, capsicum tincture, and the like), antihistamines (isothipendyl hydrochloride and the like), anti-inflammatory agents (guaiazulene and the like), keratolytics (urea, salicylic acid, and the like), antimicrobial agents (chlorhexidine gluconate, isopropylmethylphenol, quaternary ammonium salts, piroctone olamine, and the like), humectants (sodium hyaluronate, chondroitin sulfuric acid, and the like), extracts of animals and plants (Taxus cuspidata, Paeonia suffruticosa, Glycyrrhisa uralensis, Hypericum erectum, aconite root, Eriobotrya japonica, Artemisia capillaris, Symphytum officinale, Angelica keiskei, Crocus sativus, Gardeniae fructus, Rosmarinus officinalis, Salvia officinalis, Saussurea lappa, Aristolochia debilis, Lupuli strobilus, placenta, and the like), vitamins (retinol acetate, pyridoxine hydrochloride, ascorbic acid, thiamin nitrate, cyanocobalamin, biotin, and the like), anti-oxidants (dibutylhydroxytoluene, sodium pyrosulfite, tocopherol, sodium edetate, ascorbic acid, isopropyl gallate, and the like), solubilizers (diisopropyl adipate, isopropyl myristate, vegetable oils, animal oils, alkyl glyceryl ethers, hydrocarbons, and the like), metabolic activators (panthenol and the like), gelling agents (water-soluble high molecular compounds and the like), adhesives, perfumes, refrigerants (mentha oil, camphor, and the like), dyes, and the like.

However, since the addition of surfactants affects the cutaneous absorption of minoxidil and decreases the feeling during use, it is preferable that the hair growth stimulant composition of the present invention does not substantially contain surfactants.

The hair growth stimulant composition of the present invention thus obtained can be used as a suitable topical preparation such as a lotion, aerosol, tonic, cream, ointment, gel, and the like.

### EXAMPLES

The present invention will be described in more detail by examples, which should not be construed as limiting the present invention.

### Example 1

3.0 g of minoxidil, 10.0 g of 1,3-butylene glycol, 35 g of ethanol, and 20 g of purified water were mixed and stirred until dissolved. A suitable amount of citric acid was added to adjust the pH of the solution to 5.5. Purified water was added again to obtain 100 ml of a lotion preparation (product of the present invention 1).

### Example 2

Using the components shown in Tables 1 and 2, lotion preparations (products of the present invention) were prepared in the same manner as in Example 1. Using the components shown in Table 3, lotion preparations (comparative products) were prepared in the same manner as in Example 1.

(Composition)

TABLE 1

| | Products of the Present Invention | | | | | |
|---|---|---|---|---|---|---|
| | 2 | 3 | 4 | 5 | 6 | 7 |
| minoxidil | 3.0 g | 5.0 g | 5.0 g | 3.0 g | 3.0 g | 5.0 g |
| 1,3-butylene glycol | 10.0 g | 20.0 g | 30.0 g | — | — | 10.0 g |
| glycerol | — | — | — | — | — | 5.0 g |
| dipropylene glycol | — | — | — | 10.0 g | — | — |
| Macrogol 400 | — | — | — | — | 10.0 g | — |
| ethanol | 35.0 g | 60.0 g | 50.0 g | 60.0 g | 40.0 g | 55.0 g |
| citric acid | — | — | — | — | — | proper amount |
| lactic acid | proper amount | — | — | — | — | — |
| phosphoric acid | — | proper amount | — | — | proper amount | — |
| hydrochloric acid | — | — | proper amount | 1.0 g | — | — |
| sodium hydroxide | — | — | — | proper amount | — | — |
| purified water | balance* | balance* | balance* | balance* | balance* | balance* |
| pH | 5.5 | 6.5 | 6.1 | 6.5 | 6.0 | 6.0 |

*The total amount of the preparation is 100 ml

TABLE 2

| | Products of the Present Invention | | | | | | | |
|---|---|---|---|---|---|---|---|---|
| | 8 | 9 | 10 | 11 | 12 | 13 | 14 | 15 |
| minoxidil | 3.0 g | 5.0 g | 3.0 g | 3.0 g | 5.0 g | 3.0 g | 3.0 g | 5.0 g |
| 1,3-butylene glycol | 7.0 g | — | 5.0 g | 8.0 g | — | — | — | — |
| glycerol | 3.0 g | 15.0 g | 5.0 g | 2.0 g | — | — | 3.0 g | 5.0 g |
| dipropylene glycol | — | 15.0 g | — | — | 20.0 g | — | 7.0 g | 10.0 g |
| Macrogol 400 | — | — | — | — | — | 10.0 g | — | — |
| ethanol | 60.0 g | 40.0 g | 60.0 g | 60.0 g | 50.0 g | 70.0 g | 60.0 g | 55.0 g |
| citric acid | — | — | — | 1.0 g | — | — | proper | — |
| | | | | | | | amount | |
| lactic acid | proper | — | — | — | — | — | — | — |
| | amount | | | | | | | |
| phosphoric acid | — | proper | — | — | proper | — | — | — |
| | | amount | | | amount | | | |
| hydrochloric acid | — | — | proper | — | — | 1.0 g | — | proper |
| | | | amount | | | | | amount |
| sodium hydroxide | — | — | — | proper | — | proper | — | — |
| | | | | amount | | amount | | |
| purified water | balance* | balance* | balance* | balance* | balance* | balance* | balance* | balance* |
| pH | 5.5 | 6.5 | 6.1 | 5.8 | 5.6 | 6.5 | 5.5 | 6.0 |

*The total amount of the preparation is 100 ml

TABLE 3

Comparative Products

| | 1 | 2 |
|---|---|---|
| minoxidil | 3.0 g | 5.0 g |
| 1,3-butylene glycol | 5.0 g | 10.0 g |
| glycerol | — | 5.0 g |
| dipropylene glycol | — | — |
| Macrogol 400 | — | — |
| ethanol | 45.0 g | 55.0 g |
| citric acid | — | — |
| lactic acid | — | — |
| phosphoric acid | — | — |
| hydrochloric acid | — | — |
| sodium hydroxide | — | — |
| purified water | balance* | balance* |
| pH | 8.8 | 9.3 |

*The total amount of the preparation is 100 ml

The total amount of the preparation is 100 ml The products of the present invention and comparative products were stored at −10° C. for one week and the degree of crystal precipitation was examined. As a result, the product of the present invention was confirmed to display no sign of crystal precipitation even though the concentration of minoxidil was high. The comparative products were confirmed to display differing degrees of crystal precipitation.

### Example 3

An aerosol was prepared by filling an aerosol can with 30 ml of the lotion prepared in Example 1 and 70 ml of dimethyl ether. In the same manner, an aerosol was prepared using the lotion of Example 2.

### Test Example 1

Lotion preparations of the product of the present invention 16 and comparative product 3 shown in Table 4 were prepared in the same manner as in Example 1. These products were stored under the following conditions and were observed for crystal precipitation for a period of one week. The results are shown in Table 5.

Storage Conditions:

Storage temperature: −10° C.

Container: 35 ml plastic bottle (polyethylene terephthalate)

Amount: 30 ml

Number of bottles used for each observation: 8

TABLE 4

| | Product of the | |
|---|---|---|
| | Present Invention | Comparative |
| | 16 | Product 3 |
| minoxidil | 5.0 g | 5.0 g |
| 1,3-butylene glycol | 10.0 g | 5.0 g |
| ethanol | 64.34 g | 64.34 g |
| phosphoric acid | 0.5 ml | 0.5 ml |
| dibutyl hydroxy toluene | 0.05 g | 0.05 g |
| purified water | balance* | balance* |
| water content of the | 18 | 18 |
| preparation (W/W) % | | |
| pH | 6.0 | 6.0 |

*The total amount of the preparation is 100 ml

TABLE 5

| | Number of bottles exhibiting precipitation | | | |
|---|---|---|---|---|
| | Initially | After 1 day | After 4 days | After 7 days |
| Product of the | 0 | 0 | 0 | 0 |
| Present Invention 16 | | | | |
| Comparative | 0 | 0 | 3 | 3 |
| Product 3 | | | | |

### Test Example 2

Lotion preparations of the products of the present invention 17-20 shown in Table 6 and comparative products 4-8 shown in Table 7 were prepared in the same manner as in Example 1. These products were stored under the following conditions and were observed for crystal precipitation for a period of one week. The results are shown in Table 8.

Storage Conditions:

Storage temperature: −10 to −16° C.

Container: 35 ml plastic bottle (polyethylene terephthalate)

Amount: 30 ml

Number of bottles used for each observation: 8

TABLE 6

| | Products of the Present Invention | | | |
|---|---|---|---|---|
| | 17 | 18 | 19 | 20 |
| minoxidil | 5.0 g | 5.0 g | 5.0 g | 5.0 g |
| 1,3-butylene glycol | 10.0 g | 20.0 g | 30.0 g | 40.0 g |
| ethanol | 49.0 g | 41.0 g | 33.0 g | 24.0 g |
| phosphoric acid | 0.5 ml | 0.5 ml | 0.5 ml | 0.5 ml |
| purified water | balance* | balance* | balance* | balance* |
| water content of the | 32 | 32 | 31 | 32 |
| preparation (W/W) % | | | | |
| pH | 5.7 | 5.7 | 5.7 | 5.7 |

*The total amount of the preparation is 100 ml

TABLE 7

| | Comparative Products | | | | |
|---|---|---|---|---|---|
| | 4 | 5 | 6 | 7 | 8 |
| minoxidil | 5.0 g | 5.0 g | 5.0 g | 5.0 g | 5.0 g |
| 1,3-butylene | 5.0 g | 10.0 g | — | — | — |
| glycol | | | | | |
| glycerol | — | 5.0 g | — | — | — |
| propylene | — | — | 5.0 g | 5.0 g | 5.0 g |
| glycol | | | | | |
| ethanol | 53.0 g | 55.0 g | 40.0 g | 60.0 g | 60.0 g |
| phosphoric | 0.5 ml | — | 0.5 ml | 0.05 ml | 0.2 ml |
| acid | | | | | |
| purified | balance* | balance* | balance* | balance* | balance* |
| water | | | | | |
| water | 33 | 24 | 53 | 28 | 28 |
| content | | | | | |
| of the | | | | | |
| preparation | | | | | |
| (W/W) % | | | | | |
| pH | 5.7 | 9.3 | 5.5 | 7.0 | 6.4 |

*The total amount of the preparation is 100 ml

TABLE 8

| | Number of bottles exhibiting precipitation | | | |
|---|---|---|---|---|
| | Initially | After 1 day | After 4 days | After 7 days |
| Product of the | 0 | 0 | 0 | 0 |
| Present Invention 17 | | | | |
| Product of the | 0 | 0 | 0 | 0 |
| Present Invention 18 | | | | |
| Product of the | 0 | 0 | 0 | 0 |
| Present Invention 19 | | | | |
| Product of the | 0 | 0 | 0 | 0 |
| Present Invention 20 | | | | |
| Comparative | 0 | 0 | 1 | 1 |
| Product 4 | | | | |
| Comparative | 0 | 1 | 1 | 1 |
| Product 5 | | | | |
| Comparative | 0 | 1 | 1 | 1 |
| Product 6 | | | | |
| Comparative | 0 | 1 | 1 | 1 |
| Product 7 | | | | |
| Comparative | 0 | 0 | 1 | 1 |
| Product 8 | | | | |

1. A hair growth stimulant composition comprising 10 to 50% by mass of water and 3% by mass or more of minoxidil, further comprising 8 to 30% by mass of polyhydric alcohol, and having a pH of 5.5 to 6.5.

2. The hair growth stimulant composition according to claim 1, wherein the polyhydric alcohol is 1,3-butylene glycol.

3. The hair growth stimulant composition according to claim 1, wherein the polyhydric alcohol is a mixture of 1,3-butylene glycol and glycerol at a weight ratio of 10:1 to 1:4.

4. The hair growth stimulant composition according to claim 1, wherein the polyhydric alcohol is a mixture of dipropylene glycol and glycerol at a weight ratio of 10:1 to 1:4.

5. The hair growth stimulant composition according to any one of claims 1-4, wherein the incorporated amount of minoxidil is 3 to 6% by mass.

6. The hair growth stimulant composition according to any one of claims 1-5 in the form of a lotion further containing ethanol.

7. The hair growth stimulant composition according to any one of claims 1-5 in the form of a gel further containing ethanol.

8. The hair growth stimulant composition according to any one of claims 1-7, wherein the incorporated amount of ethanol is 70% by mass or less.

9. The hair growth stimulant composition according to any one of claims 1-8, wherein the incorporated amount of water is 18 to 50% by mass.

10. The hair growth stimulant composition according to any one of claims 1-9 substantially not containing surfactants.

11. The hair growth stimulant composition according to any one of claims 1-10 containing citric acid, hydrochloric acid, lactic acid, or phosphoric acid as a pH adjustor.

12. The hair growth stimulant composition according to any one of claims 1-11 further comprising one or more components selected from the group consisting of menthol, vitamin E acetate, pantothenylethylether, hinokitiol, glycyrrhetic acid, and diphenhydramine hydrochloride.

13. A method for preventing precipitation which comprises incorporating 8 to 30% by mass of a polyhydric alcohol in the composition containing 10 to 50% by mass of water and 3% by mass or more of minoxidil, and adjusting the composition to a pH of 5.5 to 6.5.
